(19) United States
(12) Patent Application Publication
(10) Pub. No.: US 2004/0082652 A1
(43) Pub. Date: Apr. 29, 2004

(54) Nitric ester derivatives and their use in treating gastrointestinal tumors

(75) Inventors: Piero Del Soldato, Monza (IT); Francesco Sannicolo, Milano (IT)

Correspondence Address: ARENT FOX KINTNER PLOTKIN & KAHN, 1050 CONNECTICUT AVENUE, N.W., SUITE 400, WASHINGTON, DC, 20036, US

(73) Assignee: Nicox S.A.

(21) Appl. No.: 10/686,907

(22) Filed: Oct. 17, 2003

Related U.S. Application Data
(62) Division of application No. 09/147,770, filed on Apr. 28, 1999.
Application No. 09/147,770 is a 371 of international application No. PCT/EP97/04774, filed on Sep. 2, 1997.

(30) Foreign Application Priority Data
Sep. 4, 1996 (IT) MI96A001821

(51) Int. Cl. A61K 31/21

(52) U.S. Cl. 514/509

(57) ABSTRACT

Use of the following groups of compounds or their compositions for the preparation of medicaments for the treatment of gastrointestinal tumors, such compounds having general formula: A-X₁—NO₂ or their salts, where A=R(COX)ₜ and where t is an integer 0 or 1; X═O, NH, NR_{1c}, where R_{1c} is a linear or branched alkyl having from 1 to 10 C atoms; R is (IA) where t=1 and X₁ is equal to —YO— where Y is a C₁-C₂₀ alkylene, C₅-C₇ cycloalkyl or oxyalkyl derivatives.

The present invention relates to new medicaments to be used in urinary disorders. These disorders are generally grouped in one single functional pathology class and are characterized by several symptoms, including changes in micturition (like for example incontinence), changes in urinary output (like for example polyuria, oliguria, anuria), changes in the appearance of urine (like for example hematuria), edema (like for example anasarca), pain (like for example bladder pain).

The invention relates to new compounds having superior efficacy in the treatment of some forms of urinary incontinence (anti-incontinence compounds) or edema (diuretics) and which appear to be well tolerated by the body.

In particular, it is known that urinary incontinence cam be considered a disorder of micturition control resulting from a lesion or dysfunction of the lower urinary tract. In particular, the urinary bladder smooth muscle called detrusor muscle and the internal (smooth muscle) and external (striated muscle) urethral sphincters are involved. See for example Ferguson D. and Christopher N., Urinary bladder function and drug development, Trends in Pharmacological Sciences, 1996, 17, 161-165. This publication reports that there are various types of incontinence characterised by different causes and symptoms. In particular, the following can be mentioned:

stress incontinence, which is the discharge of small amounts of urine due to increased intraabdominal pressure caused, for example, by cough or an effort. Stress incontinence is due to a change in vesicourethral angle and relaxation of the urethral sphincter muscle. Stress incontinence is frequent in women, particular multipara women.

urge incontinence, which is the inability to control the urinary bladder and manifests itself with a sudden and urgent stimulus to urinate. Urge incontinence is due to intermittent contraction of the urinary bladder muscle for no apparent cause (detrusor instability) or caused by interstitial cystitis or other inflammatory phenomena which lead to urinary bladder hyperexcitability. It seems that changes in urinary bladder innervation are present in all these cases;

incontinence from urinary bladder overdistention, which occurs in case of chronic urinary retention due to obstructive causes. The urinary bladder never empties completely, resulting in continous discharge of small amounts of urine.

total incontinence, which is a complete lack of urinary bladder control due to inability of controlling the sphincters. It is the result of severe neurologic damages.

In the known art, the available therapies are based on three different approaches, see for example the above publication and Anderson K. E., Pharmacology of lower urinary tract smooth muscles and penile erectile tissues, Pharmacological Reviews, 1993, 45, 253-308:

reduced detrusor activity,

changed sensory nervous transmission,

changed urethral resistance.

According to the first approach, detrusor contraction is stimulated by the parasympathetic system and acetylcholine is the main mediator. Therefore, anticholinergic agents are used to reduce vesical hyperactivity. However, these are effective but of limited use due to the systemic anticholinergic side effects including for example dry mouth, constipation and tachycardia. Taking into account that vesical irritability is often associated with urinary bladder obstructive disease, the administration of anticholinergic agents risks triggering an acute urinary retention crisis.

Another pharmacological approach to reduce detrusor activity includes the use of medicaments which help opening potassium channels or calcium antagonists which relax the smooth muscle. However, there are disadvantages such as a marked hypotensive action due to a nonspecific vasodilator effect produced by these agents.

An additional pharmacological measure to reduce detrusor activity consists of the use of prostaglandin synthesis inhibitors which were tested in some detrusor hyperactivity and enuresis cases with promising results but giving major side effects. Their use is based on the fact that numerous prostaglandins were found to be synthesised in the urinary bladder following nervous stimulation and some of them seem to act as mediators of detrusor contraction. Additionally, some prostaglandines may be involved in severe urge incontinence and vesical hyperactivity events during some inflammatory disease of the urinary tract.

Therefore, nonsteroidal anti-inflammatory drugs are potentially useful in lowering the urinary bladder excitability threshold, and are thus effective in cases of detrusor instability. Unfortunately, they have the disadvantage of being little tolerated at active doses, especially in the gastrointestinal tract.

Likewise, NO synthetase enzyme inhibitors can prevent hyperexcitability of the urinary bladder and hyperalgesia resulting from inflammatory events such as interstitial cystitis; see Rice A.S.C., Topical spinal administration of a nitric oxide synthase inhibitor prevents the hyperreflexia associated with a rat model of persistent visceral pain, Neuroscience Letters, 1995, 187, 111-114. However, there are currently no agents of this type which can be used therapeutically due to a relative nonspecificity of their pharmacological profile.

The second approach, which consists of changing sensory nervous transmission (whenever urinary incontinence results from lesions of the nervous system), includes the use of drugs which act on neurotransmission, for example gamma-aminobutyric acid (GABA), or peptides, or purines, which are important neurotransmitters in the urinary tract.

Studies are also known which use capsaicin for intravesical instillation with sometimes satisfactory results. However, this treatment has limited clinical applications due to its transient effect, which, in addition, can be obtained only by local use.

The third approach considers the fact that muscle tone in the urethra is mediated by different neurotransmission systems, including for example the adrenergic system, by stimulation of α-receptors, α-agonist medicaments, which increase the pressure borne by the urethra, are used to change urethral resistance sometimes with satisfactory results. However, the use of these compounds involves some risks, as in the case of urinary bladder obstructive disease where even alpha-antagonists are used. In these cases a sphincter hyperactivity is observed, which prevents regular urinary bladder voiding causing urge incontinence. Also in this case, as in the first approach described above, severe side effects of a hypontensive type related to the α-antagonistic activity in the cardiocirculatory system are observed.

To increase urethral resistance in women with stress incontinence, an oestrogen based therapy which was found to be efficacious in increasing intraurethral pressure and in changing the structure of mucous membrane, vessel and connective, is used. Good results were observed combining treatment with α-agonists with oestrogen treatment. However, the well known side effects which occur when oestrogen treatment is used must be reported.

So far, commercial pharmaceutical preparations resolve the problem only in a limited number of cases. However, they generally cause side effects, even somewhat severe.

The Applicant has unexpectedly and surprisingly found that the particular classes of compounds described below can be beneficially used in the treatment of the various types of urinary incontinence described above, as they exhibit a pharmacological profile superior to that of the known preparations used for this type of disease.

An object of the present invention is the use for the treatment of urinary incontinence of the following classes of compounds, having the general formula:

A-X₁—NO₂

or their salts, wherein

A=R(COX)ₜ where t is an integer 0 or 1;

X═O, NH, NR_{1c} where R_{1C} is a linear or branched alkyl having from 1 to 10 C atoms;

R is chosen from the following groups:

Group IA), wherein t=1

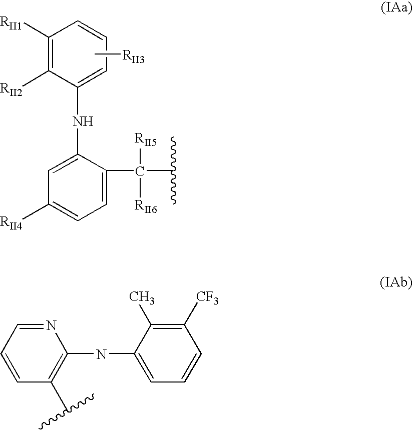

wherein

R_{II5} is H, a linear or whenever possible branched C₁-C₃, alkyl;

R_{II6} has the same meaning as R_{II5}, or when R_{II5} is H it can be benzyl;

R_{II1}, R_{II2} and R_{II3}, equal or different, are hydrogen, linear or whenever possible branched C₁-C₆ alkyl or C₁-C₆ alkoxy, or Cl, F, Br;

R_{II4}, is R_{II1} or bromine;

preferred are the compounds where R_{II1}, R_{II2} and R_{II4} are H, and R_{II3} is Cl, and R_{II3} is in the ortho position to NH; R_{II5} and R_{II6} are H, X is equal to O, and X₁ is (CH₂—CH₂—O)₂;

(IAb) is the residue of 2-[[2-methyl-3-(trifluoro-methyl)phenyl]amino]-3-pyridinecarboxylic acid and when —COOH is present it is known as flunixin.

The compounds preferred are those where X═O;

IIA) chosen from the following wherein when t=1,

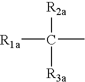

wherein

R₂ₐ and R₃ₐ are H, a linear or whenever possible branched substituted or non-substituted C₁-C₁₂ alkyl, allyl, with the proviso that when one of the two is allyl the other is H;

preferably R₂ₐ is H, alkyl has from 1 to 4 C atoms, R₃ₐ is H;

R₁ₐ is chosen from II Aa)

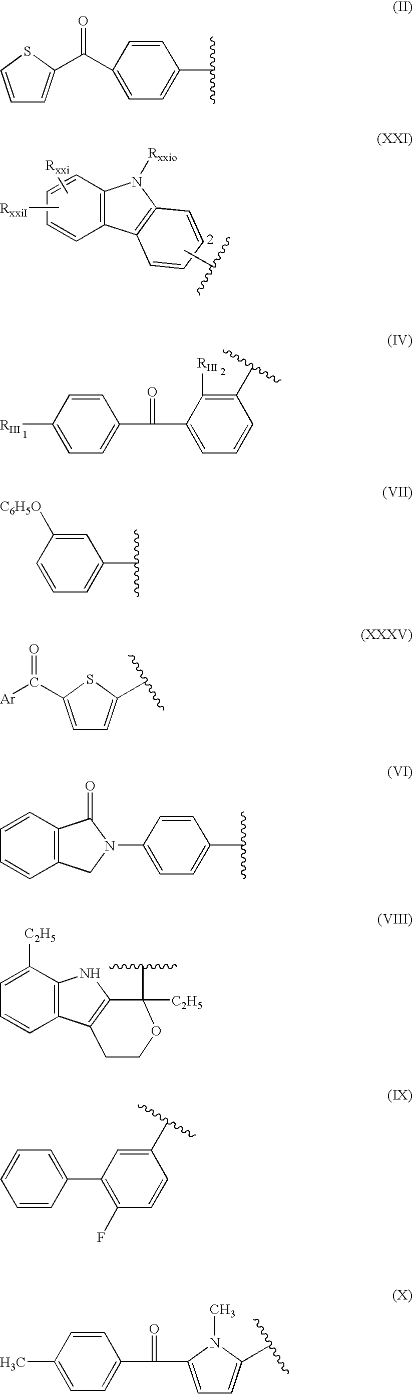

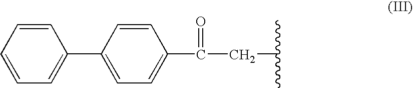

wherein:

in the compounds of formula (IV), residue of ketoprofen:

R_{III1}is H, SR_{III3} where R_{III3} contains from 1 to 4 C atoms linear or whenever possible branched;

R_{III2} is H, hydroxy;

preferred are the compounds where R_{III1} and R_{III2} are H, R₃ₐ is H, and R₂ₐ is methyl, X═C;

in the compounds of formula (XXI), residue of carprofen:

Rₓₓᵢₒ is H, a linear or whenever possible branched alkyl having from 1 to 6 carbon atoms, a C₁-C₆ alkoxycarbonyl bound to a C₁-C₆ alkyl, a C₁-C₆ carboxyalkyl, a C₁-C₆ alkanoyl, optionally substituted with halogen, benzyl or halobenzyl, benzoyl or halobenzoyl;

Rₓₓᵢ is H, halogen, hydroxy, CN, a C₁-C₆ alkyl optionally containing OH groups, a C₁-C₆ alkoxy, acetyl, benzyloxy, SRₓₓᵢ₂ where Rₓₓᵢ₂ is C₁-C₆ alkyl; a perfluoroalkyl having from 1 to 3 C atoms, a C₁-C₆ carboxyalkyl optionally containing OH, NO₂, sulphamoyl, dialkyl sulphamoyl groups with the alkyl having from 1 to 6 C atoms, or difluoroalkylsulphonyl with the alkyl having from 1 to 3 C atoms;

Rₓₓᵢ₁ is halogen, CN, a C₁-C₆ alkyl containing one or more OH, a C₁-C₆ alkoxy, acetyl, acetamide, benzyloxy groups, SR_{III3} is as above defined, a perfluoroalkyl having from 1 to 3 C atoms, hydroxy, a carboxyalkyl having from 1 to 6 C atoms, NO₂, amino, a mono- or dialkylamino having from 1 to 6 C atoms, sulphamoyl, a dialkyl sulphamoyl having from 1 to 6 C atoms, or difluoroalkylsulphamoyl as above defined; or Rₓₓᵢ together with Rₓₓᵢ₁ is an alkylene dioxy having from 1 to 6 C atoms;

preferred are the compounds where Rₓₓᵢₒ is H, the connectiong bridge is at position 2, Rₓₓᵢ is H, Rₓₓᵢ₁ is chlorine and is in para position to nitrogen;

R₃ₐ is H, R₂ₐ is methyl and X is O;

in the compounds of formula (XXXV), residue of thiaprofenic acid:

Ar is phenyl, hydroxyphenyl optionally mono or polysubstituted with halogen, an alkanoyl or alkoxy having from 1 to 6 C atoms, a trialalkyl having from 1 to 6 C atoms, preferably from 1 to 3 C atoms, cyclopentyl o-hexyl o-heptyl, heteroaryl, preferably thienyl, furyl optionally containing OH, pyridyl;

the preferred compounds of formula (XXXV) are those

where Ar is phenyl, R₃ₐ is H, R₂ₐ is methyl and X is O;

in the compounds of formula (II), residue of suprofen, of which the preferred, where R₃ₐ═H, R₂ₐ═CH₃ and X═O, has been shown, its equivalents, as described and obtained in patent U.S. Pat. No. 4,035,376, herein incorporated by reference, can be used.

in the compounds of formula (VI), of which the preferred, indoprofen, when R₂ₐ is CH₃ or indobufen, when R₂ₐ is equal to H and R₃ₐ═CH₃ and X═O, have been shown, its equivalents, as described and obtained in accordance with patent U.S. Pat. No. 3,997,669, herein incorporated by reference, can be used.

in the compounds of formula (VIII), of which the preferred etodolac, when R₃ₐ═R₂ₐ═H and X═O, has been shown, its equivalents as described and obtained according to patent U.S. Pat. No. 3,843,681, herein incorporated by reference, can be used;

in the compounds of formula (VII), of which the preferred, fenoprofen, when R₃ₐ═H, R₂ₐ═CH₃ and X═O, has been shown, its equivalents, as described and obtained according to patent U.S. Pat. No. 3,600,437, herein incorporated by reference, can be used;

in the compounds of formula (III), of which the preferred, fenbufen, when R₃ₐ═R₂ₐ═H and X═O, has been shown, its equivalents, as described and obtained according to patent U.S. Pat. No. 3,784,701, herein incorporated by reference, can be used;

in the compounds of formula (X), residue of tolmetin, when R₃ₐ═R₂ₐ═X and X═O, its equivalents, as described and obtained according to patent FR 1.574.570, herein incorporated by reference, can be used;

in the compounds of formula (IX), residue of flurbiprofen, when R₃ₐ═H, R₂ₐ═CH₃ and X═O, its equivalents as described in the known art can be used; II Ab)

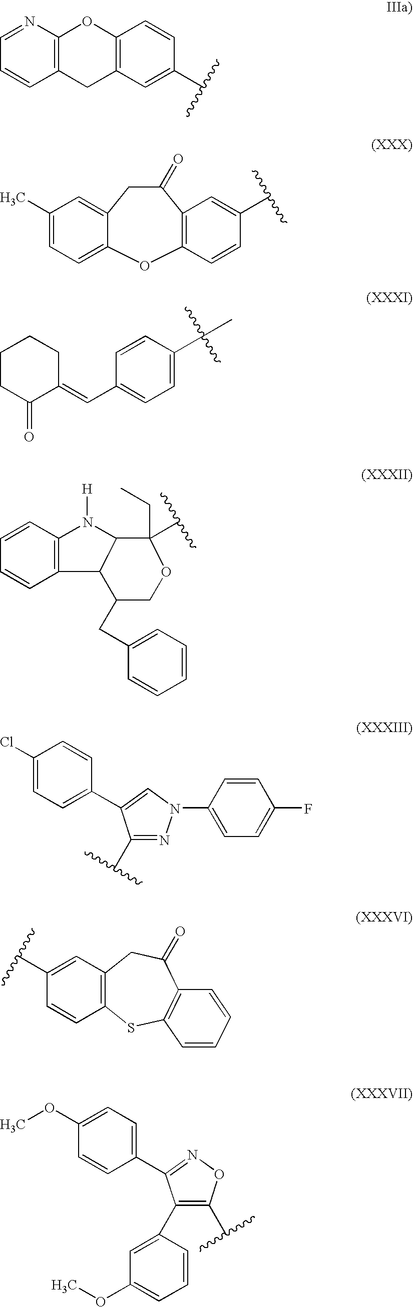

where the meanings are as follows:

when IIIa) contains —CH(CH₃)—COOH it is known as pranoprofen: α-methyl-5H-[1]benzopyran[2,3-b]pyridine-7-acetic acid. In the preferred compounds R₂ₐ═H, R₃ₐ═CH₃ and X═O;

when residue (XXX) contains —CH(CH₃)—COOH it is known as bermoprofen: dibenz[b,f]oxepin-2-acetic acid. The preferred compound is that with X═O, R₂ₐ═H, R₃ₐ═CH₃;

residue (XXXI) is known as CS-670, 2-[4(2-oxo-1-cyclo-hexylidenemethyl)phenyl]propionic acid, when the radical is —CH(CH₃)—COOH. The preferred compound has R₂ₐ═H, R₃ₐ═CH and X═O;

residue (XXXII) derives from the known pemedolac which contains group —CH₂COOH. The compound preferred has R₂ₐ═R₃ₐ═H and X═O;

when residue (XXXIII) is saturated with —CH₂COOH it is known as pyrazolac: 4-(4-chlorophenyl)-1-(4-fluorophenyl)3-pyrazolyl acid derivatives. The preferred compounds have R₂ₐ═R₃ₐ═H and X═O;

when residue (XXXVI) is saturated with —CH(CH₃)—COO— it is known as zaltoprofen. When the residue is saturated with a hydroxy or amine group or the acid salts, the compounds are known as dibenzothiepin derivatives. The compounds preferred have R₂ₐ═H, R₃ₐ═CH₃ and X═O;

when residue (XXXVII) is CH₂—COOH it derives from the known mofezolac: 3,4-di(p-methoxyphenyl)isoxazol-5-acetic acid. The preferred compounds include R₂ₐ═R₃ₐ═H, t=1, X═O.

Group III A), where t=1,

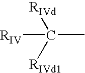

where

R_{IVd} and R_{IVd1} are at least one H and the other a linear or whenever possible branched C₁-C₆ alkyl, preferably C₁ and C₂, or difluoroalkyl with the alkyl having from 1 to 6 C atoms, preferred is C₁, or R_{IVd} and R_{IVd1} jointly form a methylene group;

R_{IV} has the following meaning:

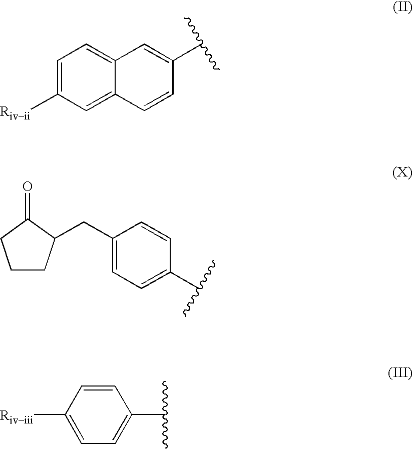

where the compounds of group III A) have the following meanings:

in the compounds of formula (II):

R_{IV-II} is an alkyl having from 1 to 6 C atoms, a cycloalkyl having from 3 to 7 C atoms, an alcoxymethyl having from 1 to 7 C atoms, a trifluoroalkyl having from 1 to 3 C atoms, vinyl, ethynyl, halogen, an alkoxy having from 1 to 6 C atoms, a difluoroalkoxy with the alkyl having from 1 to 7 C atoms, an alkoxymethyloxy having from 1 to 7 C atoms, an alkylthiomethyloxy with the alkyl having from 1 to 7 C atoms, an alkylmethylthio with the alkyl having from 1 to 7 C atoms, cyano, difluoromethylthio, a substituted phenyl- or phenylalkyl with the alkyl having from 1 to 8 C atoms;

preferably R_{IV-II} is CH₃O, R_{IVd} is H and R_{IVd1} is CH₃, and is known as the residue of naproxen;

X═NH and X₁ is equal to (CH₂)₄ or (CH₂CH₂O)₂; also preferred is the same compound where X is equal to O;

in the compounds of formula (X), for which the residue of loxoprofen has been shown, the residues described in U.S. Pat. No. 4,161,538 herein incorporated by reference can be used as equivalents; preferred are the compounds where R_{IVd} is H and R_{IVd1} is CH₃, X═NH and X₁ is equal to (CH₂)₄ or (CH₂CH₂O)₂; also preferred is the same compound where X is equal to O;

in the compounds of formula (III): R_{IV-III} is a C₂-C₅ alkyl, even branched whenever possible, a C₂ and a C₃ alkyloxy, allyloxy, phenoxy, phenylthio, a cycloalkyl having from 5 to 7 C atoms, optionally substituted at position 1 by C₁-C₂ alkyl;

preferred is the compound where R_{IV-III} is

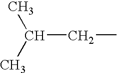

and R_{IVd}═H, R_{IVd1} is CH₃, a compound known as the residue of ibuprofen; X═NH and X₁ is equal to (CH₂)₄ or (CH₂CH₂O)₂; also preferred is the same compound where X is equal to O;

Group IV A)

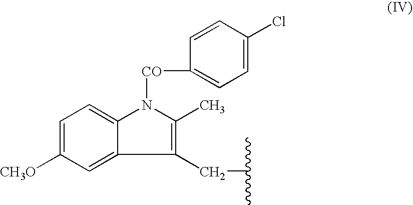

where A═RCOO, t=1,

of which the residue of the known indomethacin has been shown, its equivalents as described and obtained in patent U.S. Pat. No. 3,161,654 herein incorporated by reference can be used;

Group V A) chosen from the following:

V Aa) fenamates chosen from the following, where t=1

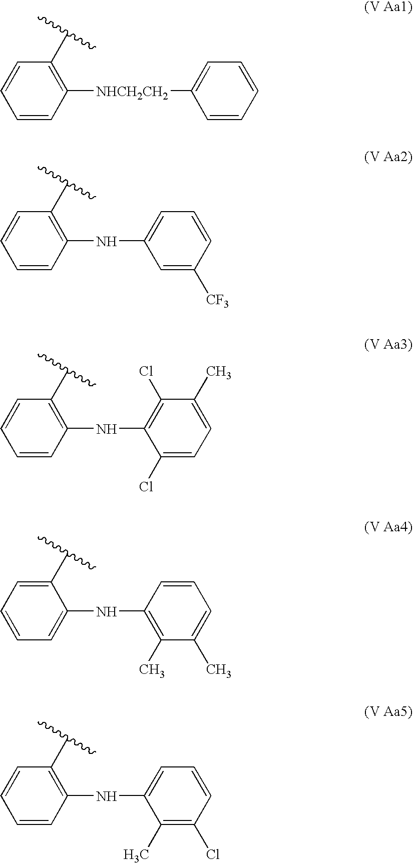

V Ab), derivatives of niflumic acid, where t=1:

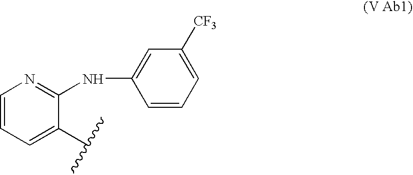

V Ac), COX₂ inhibitors, where t=0 and R is as follows:

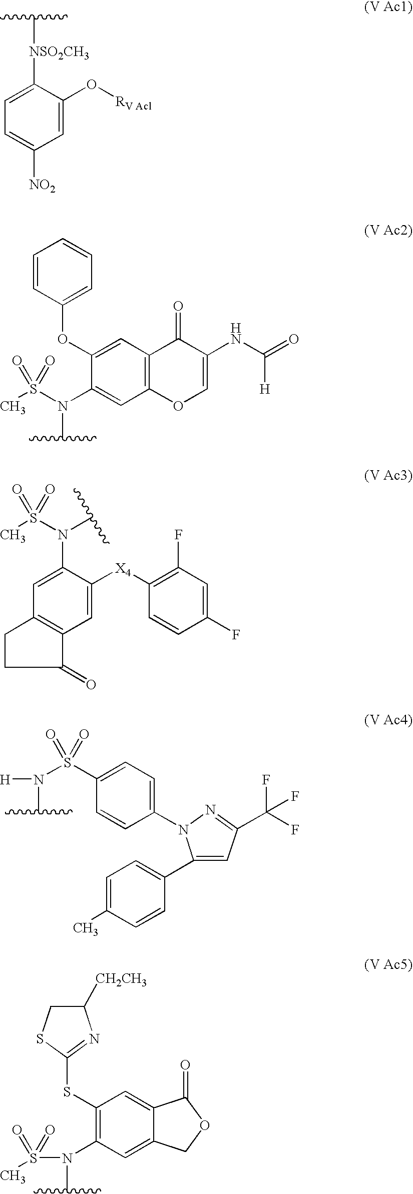

V Ad) Derivatives of diuretics when t=1 and R is as follows:

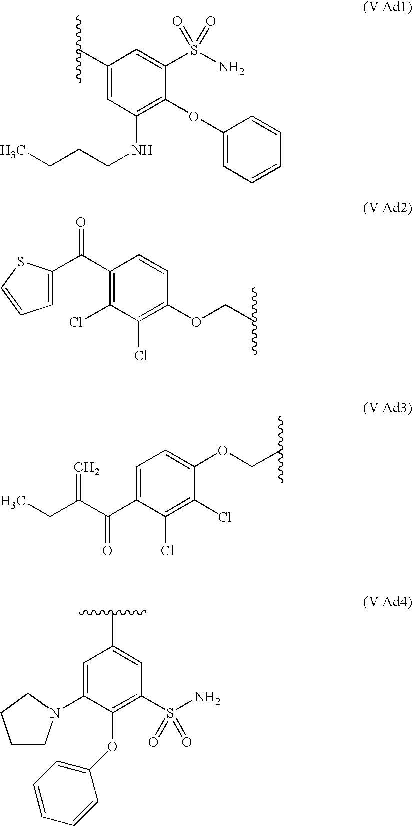

V Ae) Derivatives of diuretics when t=0 and R is as follows:

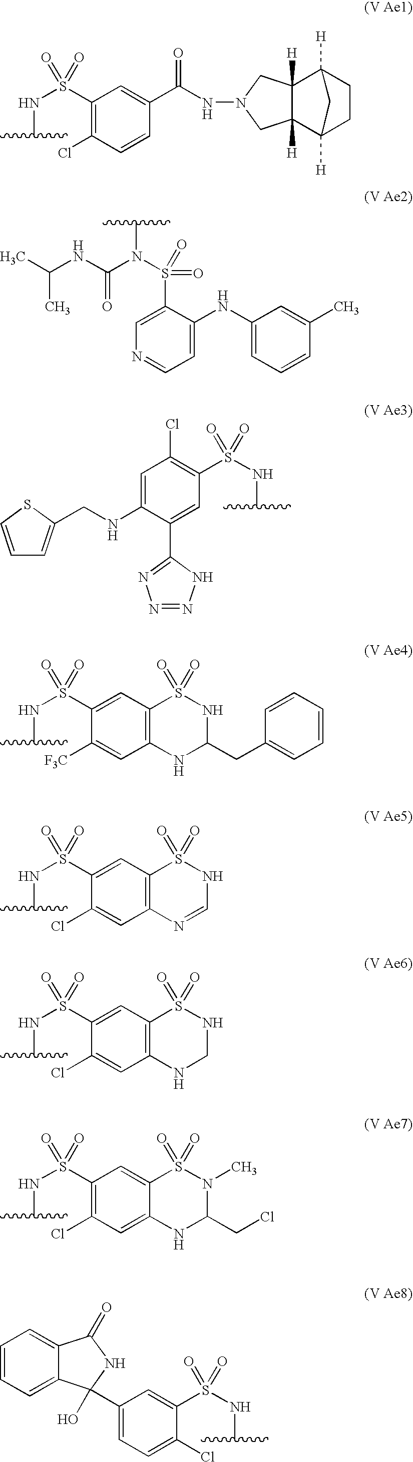

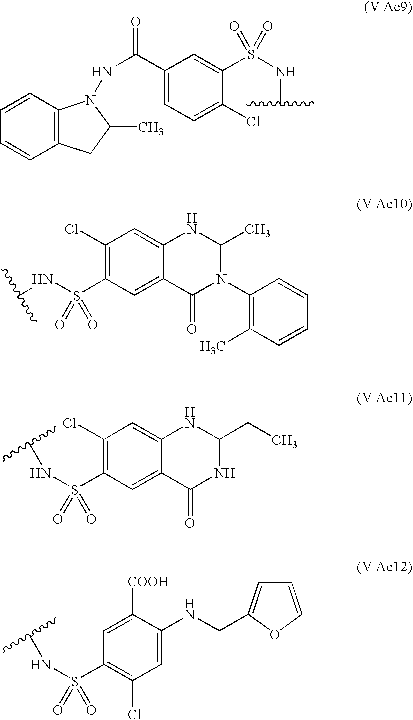

where the meaning in group V A) is as follows:

in V Aa):

in compounds (V Aa1) the residue of enfenamic acid, 2-[(2-phenylethyl)amino]benzoic acid, where COOH was substituted according to the present invention, has been shown.

This can be prepared according to the Indian patents 103.066 and 114.805, herein incorporated by reference. Equivalent products containing various substituents as described in said patents can be used, too.

In compounds (v Aa2) the residue of flufenamic acid, 2-[[3-(trifluoromethyl)phenyl]-amino]benzoic acid, where COOH was substituted according to the present invention, has been shown.

This can be prepared according to Wilkinson's article, Finar, J. Chem. Soc. 1948, 32, herein incorporated by reference. Any equivalent product containing various substituents as described in said article can be used, too.

In compounds (V Aa3) the residue of meclofenamic acid, 2-[(2,6-dichloro-3-methylphenyl)amino]benzoic acid where COOH was substituted according to the present invention, has been shown.

This can be prepared according to the German patent DE 1.149.015 and U.S. Pat. No. 3.313.848 herein incorporated by reference. Any equivalent product containing various substituents as described in said patents can be used, too.

In compounds (V Aa4) the residue of mefenamic acid, 2-[(2,3-dimethylphenyl)amino]benzoic acid where COOH was substituted according to the present invention, has been shown.

This can be prepared according to the Belgian patent 605.302, herein incorporated by reference. Any equivalent product containing various substituents as described in said patent can be used, too.

In compounds (V Aa5) the residue of tolfenamic acid, 2-[(3-chloro-2-methylphenyl)amino]benzoic acid where COOH was substituted according to the present invention, has been shown.

This can be prepared according to patent U.S. Pat. No. 3.313.848, herein incorporated by reference. Any equivalent product containing various substituents as described in said patent can be used, too.

In V Ab):

in compounds (v Ab1) the residue of niflumic acid, 2-[[3-(trifluoromethyl)phenyl]amino]-3-pyridine carboxylic acid, where COOH was substituted according to the present invention, has been shown.

This can be prepared according to patent U.S. Pat. No. 3,415,834, herein incorporated by reference. Any equivalent product containing various substituents as described in said patent can be used, too.

In V Ac):

in compound (V Ac1)R_{vac1} attached to the oxygen atom in position 2 of the benzene ring of N-(4-nitrophenyl)methansulphonamide can be phenyl or cycloexane. When R_{vac1} is phenyl the residue is that of nimesulide.

This can be prepared according to patent U.S. Pat. No. 3,840,597 herein incorporated by reference. Any equivalent product containing various substituents as described in said patent can be used, too.

In compounds (V Ac2) the residue of 3-formylamino-7-methylsulfonylamino-6-phenoxy-4H-1-bezopyran-4-one was substituted according to the present invention, has been shown.

This can be prepared according to patent DE 3834204 herein incorporated by reference. Any equivalent product containing various substituents as described in said patent can be used, too.

In compounds (V Ac3) the atom X₄ that links the radical 2,4-difluorothiophenyl to position 6 of the indanone ring of the residue 5-methanesulfonamido-1-indanone can be sulfur or oxygen.

This can be prepared according to WO 9413635 herein incorporated by reference. Any equivalent product containing various substituents as described in said patent can be used, too.

In compounds (V Ac4) the residue of celecoxib 4-[5(4-methylphenyl)-3-(trifluoromethyl)pyrazol-1-yl]benzensulphonamide was substituted according to the present invention, has been shown.

This can be prepared according to patent WO 9427980 herein incorporated by reference. Any equivalent product containing various substituents e.g. WO 9515315-318 as described in said patents can be used, too.

In compounds (V Ac5) the residue of 6-[2-(3-ethyl-2,3-dihydro-thiazolyl)thio-5-methansulphonamido-3H-isobenzonfuran-1-one was substituted according to the present invention, has been shown.

This can be prepared according to patent Wo 9623786 herein incorporated by reference. Any equivalent product containing various substituents as described in said patent can be used, too.

In compounds (V Ad1) the residue of bumetanide 3-(Aminosulfonyl)-5-(butylamino)-4-phenoxybenzoic acid was substituted according to the present invention, has been shown.

This can be prepared according to patent U.S. Pat. No. 3,806,534 herein incorporated by reference. Any equivalent product containing various substituents as described in said patent can be used, too.

In compounds (V Ad2) the residue of ticrynafen [2,3-Dichloro-4-(2-thienylcarbonyl)-phenoxy]acetic acid was substituted according to the present, has been shown.

This can be prepared according to patent U.S. Pat. No. 3,758,506 herein incorporated by reference. Any equivalent product containing various substituents as described in said patent can be used, too.

In compounds (VAd3) the residue of ethacrynic acid [2,3-Dichloro-4-(2-methylene-1-oxobutyl)phenoxy]acetic acid was substituted according to the present invention, has been shown.

This can be prepared according to patent U.S. Pat. No. 3,255,241 herein incorporated by reference. Any equivalent product containing various substituents as described in said patent can be used, too.

In compounds (V Ad4) the residue of piretanide 3-(Aminosulfonyl)-4-phenoxy-5-(1-pyrrolidinyl)benzoic acid was substituted according to the present invention, has been shown.

This can be prepared according to patent U.S. Pat. No. 4,010,273 herein incorporated by reference. Any equivalent product containing various substituents as described in said patent can be used, too.

In compounds (V Ae1) the residue of tripamide (3aα, 4α, 7α, 7aα)-3-(Aminosulphonyl)-4-chloro-N-octaidro-4,7-methano-2H-isoindol-2-yl)benzamide was substituted according to the present invention, has been shown.

This can be prepared according to patent JP 73 05, 585 herein incorporated by reference. Any equivalent product containing various substituents as described in said patent can be used, too.

In compounds (V Ae2) the residue of torsemide N-[[(1-Methylethyl)amino]carbonyl]4-[(3-methylphenyl)amino]-3-pyrinesulfonamide was substituted according to the present invention, has been shown.

This can be prepared according to patent U.S. Pat. No. 4,018,929 herein incorporated by reference. Any equivalent product containing various substituents as described in said patent can be used, too.

In compounds (V Ae3) the residue of azosemide 2-Chloro-5-(1H-tetrazol-5-yl)-4-[(2-thienylmethyl)amino]benzensulphonamide was substituted according to the present invention, has been shown.

This can be prepared according to patent U.S. Pat. No. 3,665,002 herein incorporated by reference. Any equivalent product containing various substituents as described in said patent can be used, too.

In compounds (V Ae4) the residue of bendroflumethiazide 3,4-Dihydro-3-(phenyl-methyl)-6-(trifluoromethyl)-2H-1,2,4-benzothiadiazine-7-sulfonamide 1,1-dioxide was substituted according to the present invention, has been shown.

This can be prepared according to patent U.S. Pat. No. 3,392,168 herein incorporated by reference. Any equivalent product containing various substituents as described in said patent can be used, too.

In compounds (V Ae5) the residue of chlorothiazide 6-Chloro-2H-1,2,4-benzothiadizine-7-sulfonamide 1,1-dioxide was substituted according to the present invention, has been shown.

This can be prepared according to patent U.S. Pat. No. 2,809,194, U.S. Pat. No. 2,937,169 herein incorporated by reference. Any equivalent product containing various substituents as described in said patent can be used, too.

In compounds (V Ae6) the residue of hydrochlorotiazide 6-Chloro-3,4-dihydro-2H-1,2,4-benzothiadiazine-7-sulfonamide 1,1-dioxide was substituted according to the present invention, has been shown.

This can be prepared according to patent DE 1,163,332, U.S. Pat. No. 3,043,840 herein incorporated by reference. Any equivalent product containing various substituents as described in said patent can be used, too.

In compounds (V Ae7) the residue of methyclothiazide (6-Chloro-3-(chloromethyl)-3,4-dihydro-2-methyl-2H-1,2,4-benzothiadiazine-7-sulfonamide 1,1-dioxide was substituted according to the present invention, has been shown.

This can be prepared according to patent Close et al., J. Am. Chem. Society 82, 1132 (1960) herein incorporated by reference. Any equivalent product containing various substituents as described in said patent can be used, too.

In compounds (V Ae8) the residue of chlorthalidone 2-Chloro-5-(2,3-dihydro-1-hydroxy-3-oxo-1H-isoindol-1-yl)benzensulfonamide was substituted according to the present invention, has been shown.

This can be prepared according to patent U.S. Pat. No. 3,055,904 herein incorporated by reference. Any equivalent product containing various substituents as described in said patent can be used, too.

In compounds (V Ae9) the residue of Indapamide 3-(Aminosulfonyl)-4-chloro-N-(2,3-dihydro-2-methyl-1H-indol-1-yl)benzamide was substituted according to the present invention, has been shown.

This can be prepared according to patent U.S. Pat. No. 3,565,911 herein incorporated by reference. Any equivalent product containing various substituents as described in said patent can be used, too.

In compounds (v Ae10) the residue of metolazone 7-Chloro-1,2,3,4-tetrahydro-2-nethyl-3-(2-methylphenyl)-4-oxo-6-quinazolinesulfonamide was substituted according to the present invention, has been shown.

This can be prepared according to patent U.S. Pat. No. 3,360,518 herein incorporated by reference. Any equivalent product containing various substituents as described in said patent can be used, too.

In compounds (V Ae11) the residue of quinetazone 7-Chloro-2-ethyl-1,2,3,4-tetrahydro-4-oxo-6-quinazolinesulfonamide was substituted according to the present invention, has been shown.

This can be-prepared according to patent U.S. Pat. No. 2,976,289 herein incorporated by reference. Any equivalent product containing various substituents as described in said patent can be used, too.

In compounds (V Ae12) the residue of furosemide 5-(Aminosulfonyl)-4-chloro-2-[(2-furanylmethyl)amino]benzoic acid was substituted according to the present invention, has been shown.

This can be prepared according to patent U.S. Pat. No. 3,058,882 herein incorporated by reference. Any equivalent product containing various substituents as described in said patent can be used, too.

The compounds under V Ad/V Ae are particularly suitable for the treatment of urinary disorders, in particular of anuresis.

X₁, in formula A-X₁—NO₂, is a bivalent connecting bridge chosen from the following:

YO

where Y is a linear or whenever possible branched C₁-C₂₀ alkylene, preferably having from 2 to 5 carbon atoms, or an optionally substituted cycloalkylene having from 5 to 7 carbon atoms;

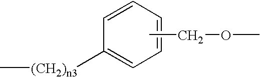

where n₃ is an integer from 0 to 3;

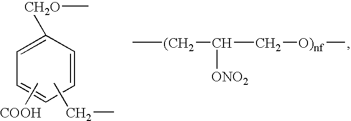

where nf′ is an integer from 1 to 6, preferably from 2 to 4;

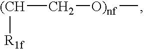

wherein R_{1f}═H, CH₃ and nf is an integer from 1 to 6 , preferably from 2 to 4.

The processes for obtaining the compounds which contain R from groups I A-IV A are described in patent application WO 95/30641 herein incorporated by reference.

The processes for preparing the compounds of class V A are those described above in application WO 95/30641.

The products of the invention are therapeutically useful in the treatment of various forms of urinary incontinence at lower doses than the corresponding precursor products without the No donor group and with a wider activity spectrum and without causing the disadvantage previously described for this kind of precursors.

It has been surprisingly found by the Applicant that, meaningfully, the products of the invention do not show reduced pharmacological activity compared to precursors. Conversely, they have a wider pharmacological range of action, since a synergy between the cyclooxigenase inhibiting effect and the myorelaxing effect related to the opening of potassium channels and/or release of nitric oxide, was unexpectedly observed in the lower urinary tract. The products of the invention exhibit a higher safety and do not induce tachyphylaxis.

Additionally, the Applicant found that the products of the invention carry out a pharmaco-therapeutic activity in diverse appropriate experimental models, as described below:

articular inflammation (musculoskeletal disease) in rats; see Winter C. et al., Caraggeenin-induced edema in hind paw of the rat as an assay for antiinflammatory drugs, Proceedings of the Society for Experimental Biology and Medicine 1962, 111, 544-47;

respiratory disease for example bronchospasm from bradykinin in Guinea pigs (Del Soldato P. et al., The anesthetized Guinea pig as a versatile pharmacological test object, Jour. of Pharmacological Methods, 1981 5, 279-285);

vascular disease, such as re-stenosis induced in rats (Role of kinins and nitric oxide in the effects of anigiotensin converting enzyme inhibitors on neointima formation, Fahry-RD et al., CIRC-RES. 72/6 (1202-1210)1983);

gynaecological and obstetrical diseases: as shown in hyperexcitability states in rat isolated myometrium (Izumi H. et al., Gestational changes in L-arginine-induced relaxation of pregnant rat and nonpregnant myometrial contractility, Am. J. Obstet. Gynecol., 1993, 169, 1327-1337);

blood platelet aggregation in women in a pre-eclampsia condition (Janes S1 et al., Flow cytometric detection of circulating activated platelets and platelet hyper-responsiveness in pre-eclampsia and pregnancy, Clin. Science, 86, 731-739, 1994).

intestinal tumours, such as for example in experimental adenocarcinoma in rats (Dubois R. et al., Increased cyclooxigenase-2 levels in carcinogen-induced rat colonic tumors, Gastroenterology, 110,1259-1262, 1996).

Therefore, based on the experimental results obtained the above products may be therapeutically useful in the following diseases, in addition to urinary incontinence:

musculoskeletal disease of-an inflammatory nature: group V A;

respiratory disease, for example bronchitis, in particular asthma, etc.: compounds of the groups from I A to V A;

gynaecological and obstetricial diseases, including premature delivery, pre-eclampsia and dysmenorrhoea: groups from I A to V A and, additionally, the compounds from group VI A as defined below;

vascular disease such as re-stenosis: compounds from groups I A to VI A;

gastrointestinal tumour: compounds from groups from I A to VI A.

The compounds in group VI A, where t=1, include the following:

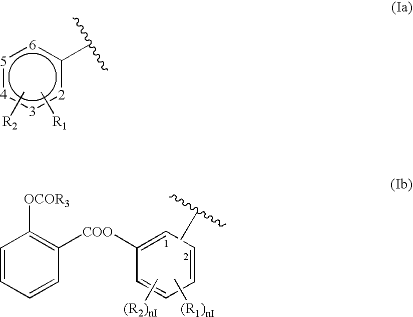

where:

R₁ is group OCOR₃; where R₃ is methyl, ethyl or a linear or branched C₃-C₅ alkyl, or the residue of a single-ring heterocycle having 5 or 6 atoms which can be aromatic, partially or totally hydrogenated, containing one or more heteroatoms independently chosen from O, N and S;

R₂ is hydrogen, hydroxy, halogen, a linear or whenever possible branched alkyl having from 1 to 4 C atoms, a linear or whenever possible branched alcoxyl having from 1 to 4 C atoms; a linear or whenever possible branched perfluoroalkyl having from 1 to 4 C atoms, for example trifluoromethyl, nitro, amino, mono-or di(C₁₋₄)alkylamino;

R₁ and R₂ jointly are the dioxymethylene group, with the proviso that when X═NH, then X₁ is ethylene and R₂═H; R₁ cannot be OCOR₃ at position 2 when R₃ is methyl; in being an integer from 0 to 1;

preferably in Ia), X is equal to O or NH, R₁ is acetoxy, preferably at position 3 or 4, most preferably in the ortho position to CO. X₁ is ethylene or (CH₂CH₂O)₂, R₂ is hydrogen or halogen, most preferred are the following A X₁ NO₂ compounds: 3-acetoxy-N-(2-nitroxyethyl)-benzamide, 4-acetoxy-N-(2-nitroxyethyl)-benzamide, 3-acetoxy-N-(5-nitroxypenthyl)-benzamide, 2-acetoxy-N-(5-nitroxypenthyl)-benzamide, N-2-(nitroxyethyl)-2-propionoxybenzamide, 2-acetoxy-2-nitroxyethylbenzoate, 2-acetoxy-N-(cis-2-nitroxycyclohexyl)-benzamide, 2-acetoxy-4-chloro-N-(2-nitroxyethyl)-benzamide, N-(2-nitroxyethyl)-2-((4-thiazolindinyl)carbonyloxy)-benzamide hydrochloride, 2-nicotinoyloxy-N-(2-nitroxyethyl)-benzamide, 2-acetoxy-5-nitroxypenthylbenzoate;

preferably in Ib) R₃═CH₃, nI=0;

X is equal to O, X₁ is ethylene; in this case Ib) is the residue of acetylsalicylsalicylic acid.

The processes to obtain the compounds which contain R in group VI A are described in patent WO 95/30641 herein incorporated by reference.

The examples below are intended as an explanation not a limitation of the present invention.

### EXAMPLES

### Examples 1,2,3 and from 1A to 1F (Comparison)

### Chemical Synthesis

The following compounds were prepared: NO-indomethacin (NO-I), NO-flufenamic (NO-F), NO-nimesulide (NO-M), NO-Naproxen (NO-N).

### Preparation of NO-Indomethacin (NO-I)

| | | |
|---|---|---|
| 3-Hydroxybenzylnitrate | 9.5 | g |
| Indomethacin | 7.4 | g |
| Dicyclohexylcarbodiimide | 5.6 | g |
| CH₂Cl₂ | 200 | ml |

were reacted and the solution was allowed to react overnight at zoom temperature, concentrated to a small volume and filtered. The filtrate was dried and passed through a column containing gel by using chloroform/ethyl acetate 14:1 as an eluting system. A head fraction was thus separated and purified by chromatography using a 2-mm silica plate. Each plate was run three times in a mobile phase made up of cyclohexane/ethyl acetate 6:1.

A yield of 85% was obtained of indomethacin-NO in group IV A where R is residue (IV) of indomethacin; t=1; X═O and X₁ is the connecting bridging, shown after YO, where n3=0, and having the general formula:

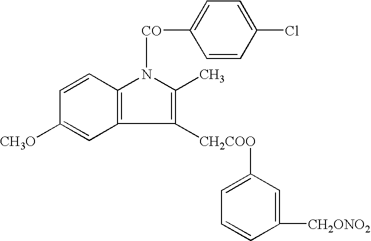

### Preparation of NO-Flufenamic (NO-F)

| | | |
|---|---|---|
| 3-Hydroxybenzylnitrate | 6 | g |
| Flufenamic acid | 13 | g |
| Dicyclohexylcarbodiimide | 9.5 | g |
| CH₂Cl₂ | 150 | ml |
| Ethyl ether | 50 | ml |

were reacted and it was allowed to react overnight, concentrated to a small volume and dicyclohexylurea was filtered. The filtrate was dried and passed through a column containing silica by using CH₂Cl₂ as an eluant. A head fraction was thus separated. This fraction was purified by chromatography using a 2-mm silica plate and a cyclohexane/ethyl acetate 6:1 system. Each plate was run three consecutive times. The head fraction was recovered by extraction with ethyl ether. The ethereal extract was brought to dryness and gives a yellow oil and a yield of 80% for flufenamic-NO.

The ¹H NMR analysis (CDCl₃, 200 MHz) gave the following data: 5.5 (2H, s); 6.9 (1H, t); 7.4 (10H, m); 8.2 (1H, dd). The product obtained-has the formula:

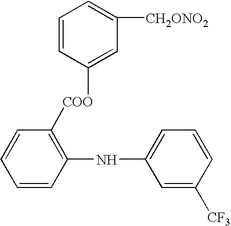

### Preparation of No-Nimesulide (NO-M)

### Preparation of the Brominated Derivative

### N-[(2-PHENOXY-4-NITRO)PHENYL]-N-(6-BROMO)HEXANOYLMETHANE-SULPHONAMIDE

4,85 g 6-Bromohexanoylchloride (23 mmol) was added dropwise to a mixture kept at 0° C. of 7 g nimesulide (23 mmol) and 6.4 ml triethylamine (46 mmol) in dichloromethane (80 ml). After stirring for one hour at 0° C., a thin layer chromatography analysis (eluant: toluene/ethyl acetate 9:1) showed that unreacted nimesulide was still present. 1 g acyl chloride (4.7 mmol) and 3 ml triethylamine (22 mmol) were added to the reaction mixture, the temperature was allowed to rise to room temperature and the reaction mixture was stirred overnight. A chromatographic control showed that the reaction was complete. The reaction mixture was treated with water (50 ml), the organic phase was then washed three times with water (50 ml for each washing), then with diluted NₐCH (5% w/v), then dried over anhydrous sodium sulphate (10 g). Solvent evaporation at reduced pressure left a yellow solid residue which was ground twice with two portions of ethyl ether (50 ml each). The air-dried solid was 8.3 g, which corresponds to a yield of 74% and exhibited a melting point of 98° C.

### Preparation of NO-Nimesulide (NO-M)

### N-[(2-PHENOXY-4-NITRO)PHENYL]-N-(6-NITROXY)HEXANOYLMETHANE-SULPHONAMIDE

A solution of 4 g N-[(2-phenoxyl-4-nitro)phenyl]-N-(6-bromo)hexanoyl-methanesulphonamide (8.24 mmol) and 2.8 g silver nitrate (16.48 mmol) in anhydrous acetonitrile (20 ml) was reacted with stirring for 2 days. Then 1 g of silver nitrate (6 mmol) was then added and stirring was continued for another day. The precipate was removed by filtration and the solvent was evaporated from the filtrate at reduced pressure. The residue was dissolved in a mixture of equal proportions of ethyl acetate and isopropyl ether and stirred for a few minutes with chromatographic-grade silica gel (5 g). The solid was removed by filtration and the filtrate of the solvent was removed at reduced pressure. The residue was a yellow oil which solidified in time (2.6 g) The solid was ground with ethyl ether and dried, and exhibited a melting point of 96° C.

The ¹H NMR spectrum (CDCl₃) showed the following signals: 8.05 (1 H, m); 7.62 (2 H, m); 7.48 (2 H, m); 7.32 (1 H, m); 7.08 (2 H, m); 4.40 (2 H, t); 3.40 (3 H, s); 2.24 (2 H, t); 2.18 (3 H, s); 1.70 (4 H, m); 1.45 (2 H, m).

### Preparation of Compound NO-Naproxen (NO-N)

Compound NO-Naproxen was prepared according to Example 1h (Example 1) in patent WO 95/30641.

### Pharmacological Tests

The products were administered in a suspension of carboxymethyl cellulose in in-vivo experiments, while they were dissolved in dimethylsulphoxide in in-vitro studies.

The same vehicle used in the corresponding treatment groups was always used for control groups.

The acute toxicity was roughly determined administering an oral dose of 50 mg/kg of substance to groups of 10 mice. Death rate and appearance of toxic symptoms were evaluated in a period of 14 days from dosing: no toxic effects were observed at the dose administered.

### Contraction Inhibiting Activity in Isolated Rat Detrusor

Male Wistar rats weighing 200 to 300 g were used. The method used is described by Zhou Q. et al. (1995) (see Example 13). After sacrificing the rats by cervical displacement the urinary bladder was isolated and horizontal strips of detrusor muscle about 2 mm wide and about 5 mm long were obtained from the median region. The strips were placed in baths for isolated organs containing Krebs liquid and subjected to a 1 g tension. Tension variations during the test were measured isometrically by using a pressure transducer connected to a-polygraph. The inhibitory effect of a pre-treatment with the test derivatives on contraction induced by 40 mM KCl was determined versus drugs having an opening potassium channel activity (cromakalin, nicorandil) nitroderivatives (nitroglycerin, nicorandil) and antiinflammatories (indomethacin, naproxen, nimesulide). The results are shown in Table 1.

TABLE 1

| Example | Product | No. of tests | Inhibition % |
|---|---|---|---|
| 1A comparison | Cromakalim 10⁻⁶ M | 10 | 33.3 |
| 1B comparison | Nitroglycerin 10⁻⁵ M | 10 | 28.7 |
| 1C comparison | Nicorandil 10⁻⁴ M | 10 | 26.4 |
| 1D comparison | Indomethancin 10⁻⁴ M | 10 | 38.5 |
| 1E comparison | Naproxen 5 · 10⁻⁴ M | 10 | 15.2 |
| 1F comparison | Nimesulide 10⁻⁴ M | 10 | 41.8 |
| 1 | NO-I 10⁻⁴ M | 10 | 46.3 |
| 2 | NO-N 5 · 10⁻⁴ M | 10 | 31.3 |
| 3 | NO-M 10⁻⁴ M | 10 | 48.1 |

All new nitroderivatives (Examples 1 to 3) proved to be more active than the products used as comparison.

### Examples 4-5 and 4A-4C (Comparison)

### In Vivo Studies in Normal Urinary Bladder of Conscious Rats

Cystometrograms of conscious rats were determined according to the method described by Howe B. B et al. (1995) (see Example 9).

Male Wistar rats weighing about 500 g were used. The rats were anaesthetised wits Nembutal. After opening their abdomen and exposing their urinary bladder, a catheter filled with physiological solution was implanted in the urinary bladder and caused to emerge from the back of the animals. The abdominal muscle and skin were then sutured. 48 hours after surgery the animals were placed in metabolic cages and the catheters were connected to a perfusor which perfused 0.18 ml/min of a physiological solution into the urinary bladder, and to a pressure transducer in order to measure intravesical pressure. After stabilisation for 60 minutes, the animals were orally treated with the test products and urination frequency was than measured during 4 hours after dosing. Table 2 shows the results obtained expressed as a ratio versus the baseline frequency recorded before dosing (IC=interval between contractions).

TABLE 2

| | | No. of | IC treated/IC |
|---|---|---|---|
| Example | Treatment | tests | baseline |
| 4 A | Controls | 8 | 1.05 |
| 4 B comparison | Flufenamic | 8 | 1.42 |
| | acid 5 mg/kg | | |
| 4 | NO-F 5 mg/kg | 8 | 1.62 |
| 4 C comparison | Indomethacin | 8 | 1.34 |
| | 5 mg/kg | | |
| 5 | NO-I 5 mg/kg | 8 | 1.48 |

Both new derivatives (Examples 4-5) proved to be more active than the products used as comparison.

### Examples 5-6 and 5A-5B (Comparison)

### In Vivo Studies in Normal Urinary Bladders of Anaesthetised Rats

40 Sprague Dawley rats weighing about 300 g were randomly divided into 4 groups and orally treated twice a day for 4 days according to the following experimental scheme:

| | | |
|---|---|---|
| 1. | Controls: | 0.5% carboxymethyl cellulose |
| 2. | Indomethacin | 3 mg/kg |
| 3. | NO-I | 3 mg/kg |
| 4. | NO-F | 5 mg/kg |

18 hours after the last treatment, the effects on the urinary bladder voiding reflex were evaluated using the method described by Maggi C. A. et al., Prostanoids modulate reflex micturition by acting through capsaicin-sensitive afferents, European Journal of Pharmacology, 105-112, 1988.

The animals were anaesthetised with urethane, the urinary bladder was prepared for intraluminal pressure measurement. After a stabilisation period with an empty urinary bladder, this was progressively filled with a physiological solution by slow infusion (0.046 ml/min). A contraction of the urinary bladder was observed upon reflex triggering.

The volume of physiological solution and intraluminal pressure required to evoke the reflex (volume and pressure thresholds) were measured. Table 3 shows the pressure and volume threshold values after treatment, calculated considering 100 the values obtained in control animals. All tested products increased this threshold and can, therefore, be considered useful in case of detrusor instability.

TABLE 3

| | | | Pressure | Volume |
|---|---|---|---|---|
| | | No. of | threshold | threshold |
| Example | Treatment | animals | (%) | (%) |
| 5 A | Controls | 10 | 100 | 100 |
| 5 B | Indomethacin | 10 | 190 | 198 |
| 5 | NO-l | 10 | 223 | 226 |
| 6 | NO-F | 10 | 203 | 205 |

### Examples 7-8 (7A-7D as Comparison)

### In Vitro Studies in Unstable Urinary Bladder

The vesical hypertrophy model secondary to urethral obstruction in rats described by Malmgren A. et al.: Cystometrical evaluation of bladder instability in rats with intravesical outflow obstruction, The Journal of Urology, 1987, 137, 1291-1294, was used in order to evaluate the effect of the drugs on hyperactive vesical muscle.

Male Sprague Dawley rats weighing about 250 g were used. In order to obtain partial urethral obstruction, the rats were anaesthetised with Nembutal and the urinary bladder and urethra were exposed by abdominal incision. A Ligature was made around the urethra in the presence of an intraluminal cannula with 1 mm diameter. After suturing the abdominal wall the animals were stabulated for 6 weeks in order for vesical hypertrophy to start.

The in vitro experiments were conducted with the parallel use of strips obtained from normal rats and rats with vesical hypertrophy.

The in vitro urinary bladder strips were prepared as described above and the inhibition induced by the drugs on contraction induced by 1/7 Hz electrical stimulation lasting 1 msec., an above maximal voltage, produced by two platinum electrodes, was measured.

The following table shows the percentage of contraction induced by electrical stimulus in normal and hypertrophic urinary bladders in the presence of the test drugs.

TABLE 4

| Example | Product/Tissue | No. of tests | Contraction % |
|---|---|---|---|
| 7 A | Cromakalim 10⁻⁶ | 6 | 50.5 |
| | M (normal) | | |
| 7 B | Cromakalin 10⁻⁶ | 6 | 35.7 |
| | M (hypertrophic) | | |
| 7 C | Indomethacin 10⁻⁶ | 6 | 78.2 |
| | M (normal) | | |
| 7 D | Indomethacin 10⁻⁶ | 6 | 76.3 |
| | M (hypertrophic) | | |
| 7 | NO-I 10⁻⁶ | 6 | 61.5 |
| | M (normal) | | |
| 8 | NO-I 10⁻⁶ | 6 | 40.3 |
| | M (hypertropic) | | |

Differently from indomethacin, the products with an opening of potassium channel activity and the new compounds were found to be more active in inhibiting hypertrophic urinary bladder contraction than normal urinary bladder.

### Examples 9-10 and from 9A to 9B (Comparison)

### In Vivo Studies in Normal Urinary Bladder of Conscious Dogs

The cystometrogram of conscious dogs was determined in accordance with the method described by Howe B. B. et al., ZENECA ZD 6169: a NOVEL K_{ATP} Channel opener with in vivo selectivity for urinary bladder, Journal of Pharmacology and Experimental Therapeutics, 274, 884-890, 1995.

Female Beagle dogs with urinary bladder catheterised through the urethra by operating in sterile conditions, were used. Catheters were connected to a perfusor which perfused into the urinary bladder a physiological solution and to a pressure transducer in order to measure intravesical pressure. After 15 minute stabilisation, a 30 ml bolus of physiological solution was perfused into the urinary bladder in order to measure increased intravesical pressure and a series of smaller boluses were then perfused until spontaneous contractions were observed. After a period of contraction stabilisation, contracting activity was monitored for 60 minutes. The animals were then treated orally with the test products and urination frequency was then measured during 4 hours following dosing in control rats and treated rats. Table 5 shows the results obtained expressed as a ratio versus the baseline frequency recorded before dosing (IC=interval between contractions).

TABLE 5

| | | No. of | IC treated/IC |
|---|---|---|---|
| Examples | Treatment | animals | baseline |
| 9 A comparison | Controls | 5 | 1.03 |
| 9 B comparison | Cromakalim | 5 | 1.48 |
| | 0.5 mg/kg | | |
| 9 C comparison | Flufenamic | 5 | 1.42 |
| | acid 3 mg/kg | | |
| 9 | NO-F 3 mg/kg | 5 | 1.76 |
| 9 D comparison | Indomethacin | 5 | 1.25 |
| | 3 mg/kg | | |
| 10 | NO-I 3 mg/kg | 5 | 1.43 |

### Examples 11-12 and 11A-11D (Comparison)

### Relaxing Effect in Pig Urethral Smooth Muscle

The method described by Werkström et al., Factors involved in the relaxation of female pig urethra evoked by electrical field stimulation, British Journal of Pharmacology, 116, 1599-1604, 1995, was used for sample preparation. Samples of urethra were removed from female pigs about 6 months old.

The urethra was opened longitudinally and samples of smooth muscle about 1×2×6 mm in size were removed from an area about 4 mm below ureteral orifices. The samples of smooth muscle were placed in baths for isolated organs, incubated at 37° C. and subjected to a 10 mN tension and connected to a force transducer for measuring mechanical activity. After a period of balancing of about 60 minutes, the prepared samples were exposed to Krebs solution without Ca₊₊ to determine the highest relaxation level. Normal tone was then restored by adding Krebs solution. The relaxation effects of the test derivatives were then measured. The test was repeated two consecutive times for each prepared sample in order to evaluate any tachyphylaxis effects. The table below shows the relaxation percentages obtained following the two treatments with each test product, expressed considering 100% the highest relaxation determined by the medium without Ca⁺⁺.

TABLE 6

| | | No. of | Relaxation % | Relaxation % |
|---|---|---|---|---|
| Example | Product | tests | 1 | 2 |
| 11 A | Indomethacin | 6 | 1.0 | 1.2 |
| | 10⁻⁵ M | | | |
| 11 | NO-I 10⁻⁵ M | 6 | 39.3 | 37.2 |
| 11 B | Flufenamic | 6 | 12.2 | 13.2 |
| | acid 10⁻⁵ M | | | |
| 12 | NO-F 10⁻⁵ M | 6 | 45.8 | 52.1 |
| 11 C | Nitroglycerin | 6 | 32.1 | 7.3 |
| | 10⁻⁵ M | | | |
| 11 D | L-arginine | 6 | 22.7 | 12.2 |
| | 10⁻⁵ M | | | |

The results show that, while drugs with an anti-inflammatory activity such as indomethacin were practically inactive except for flufenamic acid which has itself a myorelaxing activity, and conventional NO donors, such as nitroglycerin and arginine, were active but induced tachyphylaxis, the new derivatives which are an object of the invention were active and did not induce any tachyphylaxis.

### Examples 13-15 and 13A-13B (Comparison)

### Relaxing Activity on Vessel Smooth Muscle

Male Wistar rats weighing 200 to 300 g were used. The method used is described by Zhou Q. et al. The inhibitory mechanism of nicorandil in isolated rat urinary bladder and femoral artery, European Journal of Pharmacology, 153-159, 1995. After sacrifing the rats by cervical displacement, the femoral arteries were isolated for the preparation of helicoidal strips about 1×15 mm in size, from which the endothelium was removed. The prepared strips were placed in baths for isolated organs containing Krebs liquid and subjected to a weight of 0.5 g. Tension variations during the test were isometrically measured by means of a pressure transducer connected to a polygraph. The inhibitory effect of a treatment with the test derivatives on contractions induced by 3×10⁻⁵ M phenylephrine versus reference preparations having a potassium channel opening activity and/or NO donors was measured.

The results are included in Table 7.

TABLE 7

| | | No. of | |
|---|---|---|---|
| Example | Product | tests | Inhibition % |
| 13 A comparison | Cromakalim | 10 | 54.1 |
| | 3 × 10⁻⁷ M | | |
| 13 B comparison | Nicorandil | 10 | 32.6 |
| | 10⁻⁶ M | | |
| 13 | NO-I 10⁻⁴ M | 10 | 22.2 |
| 14 | NO-N 5 · 10⁻⁴ M | 10 | 29.0 |
| 15 | NO-M 10⁻⁴ M | 10 | 19.5 |

All new compounds proved to be less active than Cromakalin and Nicorandil, even used at higher concentrations, those shown in specific models (see for example Table 6).

### Example 16-17 and 16A-16B (Comparison)

### In Vivo Gastrointestinal Safety Studies

Forty Sprague Dawley rats weighing about 300 g were ramdomly divided into 4 groups and orally treated twice a day for 4 days according to the following experimental scheme:

| | | |
|---|---|---|
| 1. | Controls: carboxymethyl cellulose (0.5% by weight): | (5 ml/kg) |
| 2. | Indomethacin | 3 mg/kg |
| 3. | NO-I | 3 mg/kg |
| 4. | NO-F | 5 mg/kg |

Eighteen hours after the last treatment the rats were sacrified to determine any gastrointestinal damage. No gross changes were observed in the gastroenteric tract of control animals.

In the animals treated with indomethacin ulceration was observed in the stomachs and, additionally, the intestines of most animals (7/19) and in some cases (3/10) even diffuse adherences. In the group treated with NO-I, only gastric ulcers were observed in 1 animal, and in the group treated with NO-F one animal with a gastric ulcer and an animal with a duodenal ulcer were found.

### Examples 18-18A and 18-B (Comparison)

### Studies of Nitroxysynthetase Activity

The nitroxy-sinthetase inhibiting activity induced by lipopolisaccharide (LPS) was determined in rat neutrofils after administration of any of the test compounds and compared with that obtained after treatment with the suspending vehicle alone (0.5% carboxymethyl cellulose, 5 ml/kg) and a product used as comparison. Briefly, Wistar rats fasted for 24 hours before treatment received one of the test compounds (10 mg/kg) intraperitoneally or the vehicle LPS (5 mg/kg) intravenously (caudal vein).

Four hours later the animals were sacrified. Blood was collected for neutrofil isolation.

The enzymatic activity was determined according to the method described by Assreuy J. et al. Feedback inhibition of nitric oxide sinthase activity by nitric oxide, British Journal of Pharmacology, 883-837, 1993.

As shown in Table 8, the test product was found to be very effective in inhibiting nitroxy sinthetase compared to the group treated with the vehicle alone and differently from the reference flufenamic.

TABLE 8

| | | DOSE | NITROXY-SYNTHE- |
|---|---|---|---|
| EXAMPLE | COMPOUND | (mg/kg/i.p.) | TASE ACTIVITY^{a} |
| 18 A | Vehicle | — | 100 |
| 18 B | Flufenamic | 10 | 98 |
| 18 | NO-F | 10 | 63 |

^{a}percentage compared to the group treated with the vehicle alone.

### Conclusions from the Whole Tests

The derivatives of the invention were found to be active in several tests aimed at determining the potential pharmacological activity controlling urination.

It should also be noted that the derivatives of the invention were also found to be effective in a broader series of tests than that in which each reference drug was found to be active, confirming the hypothesis that these derivatives are endowed with a superior overall pharmacological activity in controlling urinary incontinence.

Furthermore, the derivatives of the invention were found to be better tolerated than the reference products. They appeared to be less harmful to the stomach than the corresponding anti-inflammatory agents and less hypotensive than the standard agents with vasorelaxing activity.

The combined characteristics mentioned above make the products of the invention superior to the reference agents.

### Appendix A

### In Vivo Inhibition of Precancerous Cell Formation and In Vitro Inhibition of Cancer Cell Growth by the Compounds of the Invention

### Experiment 1

### In Vivo Assay of the Inhibiting Activity of Precancerous Cell Formation by the Compounds of the Invention

Aberrant crypt foci (ACF) are preneoplastic lesions that have been consistently observed in a number of experimental models of colon carcinogenesis. Moreover, ACF are present in the mucosa of human colon cancer, where they have been suggested to be precursor lesions from which adenomas and carcinomas develop.

Since ACF express mutations in the apc gene and the ras oncogene, these lesions have been considered early markers of colon cancer development.

The compounds assayed in this test were the following:

2-(acetyloxy)benzoic acid 3-(nitrooxymethyl)phenyl ester. The compound was prepared according to ex. 1 of WO 00/44705 (NO-Asp-1).

Aspirin.

Male wistar rats (weight 200-250 g) were randomised into n. 3 groups of 8-9 animals each at the beginning of the experiment.

Colonic adenocarcinoma was induced by sequential treatment (i.v. injection) with trinitrobenzene sulfonic acid (TNBS) and azoxymethane (AOM) according to the experimental model of D'argenio et Al., Gastroenterology 110, 1727-1734 1996.

The test drugs were dissolved in DMSO diluted in 0.5% carboxymethylcellulose.

The treated groups were administered, respectively, with a daily dose of aspirin (10 mg/Kg) and with an equimolar dose (18 mg/Kg) of NO-Asp-1.

The control group was administered with the vehicle.

The treatment with TNBS and AOM lasted 28 days. Oral drug administration was continued. Six weeks after the end of the experiment the animals were sacrificed by an overdose of pentobarbital. Laparatomy was then performed with the whole colon excision.

After flushing with 0.9% saline, rhe colon was tied at both ends with a silk suture and filled with 10% phosphate buffered formalin (pH 7.4). After 2 hours the colon specimen was opened by cutting along the mesenteric border and pinned flat. Colon mucosa was then dipped into formalin.

After this treatment and rinsing from formalin, tissues were stained with 0.2% methylene blue in 0.9% saline. After 15 minutes the tissues were recovered and the number of ACF in the entire colon specimen, using a dissecting microscope at 40χ magnification. ACF were clearly identified as abnormally dilated crypts, with multiple adjacent crypts, often appearing to be contiguous.

Administration of TNBS and AOM in the control group resulted in the development of widespread precancerous cells formation in the distal colon.

Data are reported in Table 1, wherein the number of ACF cells developed in the colon of the animals of the control group was assumed to be 100%. The Table shows that NO-Asp-1 is more effective than aspirin in preventing colon neoplastic lesion.

TABLE 1
In vivo- inhibition of precancerous cell formation by
NO-Asp-1 and aspirin in an experimental model of colon
Adenocarcinoma

| | | Precancerous cell |
|---|---|---|
| | | Number |
| Compound | Dose (mg/Kg) | (% to the control group) |
| Vehicle | — | 100 |
| NO-Asp-1 | 18 | 15 |
| Aspirin | 10 | 40 |

### Experiments 2-A and 2-3

### In vitro Assay of the Antiproliferative Activity of the Compounds of the Invention in Cancerous Cells

### Experiment 2-A

Human adenocarcinoma (HT29) cells taken from colon affected by cancerous process were transferred into plates with 24 wells containing a cellular culture medium formed by 10% of foetal bovine serum, penicillin (50 U/ml), streptomycin (50 mg/ml) and PEG 400 (polyethylenglycol).

The compounds tested have been the following:

2-(acetyloxy)benzoic acid 3-(nitrooxymethyl)phenyl ester. The compound was prepared according to ex. 1 of WO 00/44705 (NO-Asp-1).

2-(acetyloxy)benzoic acid 4-(nitrooxymethyl)phenyl ester The compound was prepared according to ex. 3 of Wo 00/44705 (NO-Asp-2).

2-(acetyloxy)benzoic acid 2-(nitrooxymethyl)phenyl ester The compound was prepared according to ex. 2 of Wo 00/44705 (NO-Asp-3).

Aspirin.

After 24 hours a portion of the plates was inoculated with the tested compounds dissolved in the carrier (PEG 400). 96 hours after the inoculation of the compounds the cellular growth was measured by haemocytometer. The results, reported in Table 2, are expressed as percentage of the cellular proliferation with respect to the controls.

The obtained results show that the compounds of the invention are much more effective in inhibiting the proliferation of the cancerous cells with respect to the corresponding native compound.

TABLE 2
Activity in vitro on the proliferation of cancerous cells

| | Concentration | Proliferation |
|---|---|---|
| Treatment | (μM) | % |
| Vehicle | — | 100 |
| Aspirin | 500 | 100 |
| NO-Asp-1 | 300 | 40 |
| NO-Asp-2 | 10 | 0 |
| NO-Asp-3 | 20 | 50 |

### Experiment 2-B

In this experiment flurbiprofen (4-nitrooxy)butyl ester (NO-flurbiprofen) and flurbiprofen, as reference compound, were used.

Flurbiprofen (4-nitrooxy)butyl ester was obtained according to Ex. 1 of WO 94/12463.

HT-29 and HCT-15 human colon adenocarcinoma cell lines (American Type Culture Collection) were grown as monolayers in McCoy 5A medium and RPMI 1640 respectively, and supplemented with 10% foetal calf serum (FCS), penicillin (50 U/ml) and streptomycin (50 mg/ml). Cells were seeded at a density of 1.5 million cells/100 cm² culture dish and incubated at 37° C. in 5% CO₂ and 90% relative humidity. Single-cell suspensions were obtained by tripsinization (0.05% trvosin/EDTA), and cells were counted using a hemacytometer.

Viability was determined by the tryptan blue dye exclusion method.

The compounds under test were dissolved in dimethyl sulfoxide (DMSO) solutions. All compounds were added to the culture medium before plating. Final DMSO concentration was adjusted in all media to 1% w/v.

A control group was run by adding the the cells a same quantity of DMSO.

NO-flurbiprofen reduced the number of HT-29 cells in the culture more effectively than flurbiprofen. Similar results were obtained with HCT-15 cells.

48 hours after incubation with the compounds, the cells were counted using a hemacytometer. Table 3 reports the results obtained, expressed as percentage of cellular growth with respect to the control group.

TABLE 3
Activity in vitro on the proliferation of cancerous cells

| | Concentration | |
|---|---|---|
| Treatment | (μM) | Growth at 48 hours |
| Vehicle | — | 100 |
| Flurbiprofen | 500 | 55 |
| NO-Flurbiprofen | 500 | 30 |

What is claimed:

1. A method for treatment of gastrointestinal tumors by administering compounds, having the formula:
A-X₁—NO₂
or their salts, where:
A=R(COX)ₜ wherein t is an integer 0 or 1;
X═O, NH, NR_{1C} wherein R_{1C} is a linear or branched alkyl having from 1 to 10 C atoms;
R is chosen from the following groups:
Group I A), where t=1, 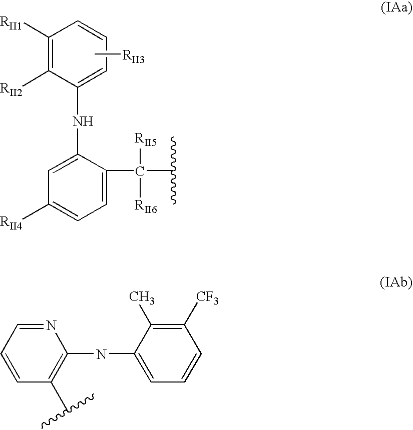
where:
R_{II5} is H, a linear C₁-C₃ alkyl, or a branched C₁-C₃ alkyl;
R_{II6} has the same structure as R_{II5},
R_{II1}, R_{II2} and R_{II3} are each hydrogen, linear C₁-C₆ alkyl, branched C₁-C₆ alkyl, C₁-C₆ alkoxy, Cl, F, or Br;
R_{II4} has the same structure as R_{II1}, or is bromine;
Group 11 A) chosen from the following:
where, when t=1, R is 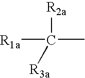
where R₂ₐ and R₃ₐ are H, a linear C₁-C₁₂ alkyl, a branched C₁-C₁₂ alkyl, or allyl, with the proviso that when one of the two is allyl the other is H;
R₁ₐ is chosen from the subgroup II Aa) consisting of 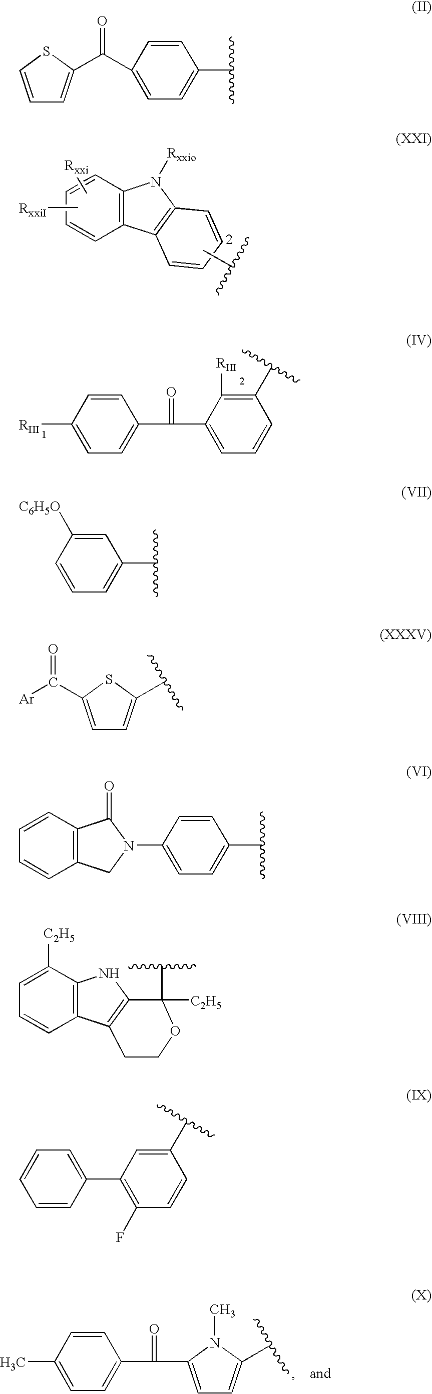 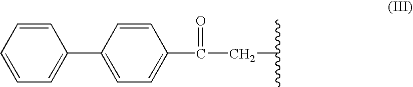
wherein:
in the residue of formula (IV):
R_{III1} is H or SR_{III3} where R_{III3} contains from 1 to 4 linear or branched C atoms; and
R_{III2} is H or hydroxy;
in the residue of formula (XXI):
Rₓₓᵢₒ is H, a linear alkyl having 1-6 carbon atoms, a branched alkyl having from 1 to 6 carbon atoms, a C₁-C₆ alkoxy-carbonyl bound to a C₁-C₆ carboxyalkyl, or a C₁-C₆ alkanoyl, optionally substituted with halogen, benzyl or halobenzyl, benzoyl or halobenzoyl;
Rₓₓᵢ is H, halogen, hydroxy, CN, a C₁-C₆ alkyl optionally containing OH groups, a C₁-C₆ alkoxy, acetyl, benzyloxy, SRₓₓᵢ₂ where Rₓₓᵢ₂ is a C₁-C₆ alkyl; a perfluoroalkyl having a 1-3 C atoms, a C₁-C₆ carboxyalkyl optionally containing OH groups, NO₂, sulphamoyl, dialkyl sulphamoyl with the alkyl having from 1 to 6 C atoms, or difluoroalkylsulphonyl with the alkyl having from 1 to 3 C atoms;
Rₓₓᵢ₁ is halogen, CN, a C₁-C₆ alkyl optionally containing one or more OH groups, a C₁-C₆ alkoxy, acetyl, acetamido, or benzyloxy, SR_{III3} is as above defined, a perfluoroalkyl having from 1 to 3 C atoms, hydroxy, a carboxyalkyl having from 1 to 6 C atoms, hydroxy, a carboxyalkyl having from 1 to 6 C atoms, NO₂, amino, mono- or dialkylamino having from 1 to 6 C atoms, sulphamoyl, a dialkyl sulphamoyl having from 1 to 6 C atoms, difluoroalkylsulphamoyl; or Rₓₓᵢ together with Rₓₓᵢ₁ is an alkylene dioxy having from 1 to 6 C atoms;
In the residue of formula (XXXV):
Ar is phenyl, hydroxyphenyl optionally mono- or polysubstituted with halogen, an alkanoyl or alkoxy having from 1 to 6 C atoms, a trialalkyl having from 1-6 C atoms, cyclopentyl o-hexyl o-heptyl, thienyl, furyl, furyl containing OH, or pyridyl;
Subgroup II Ab) consisting of: 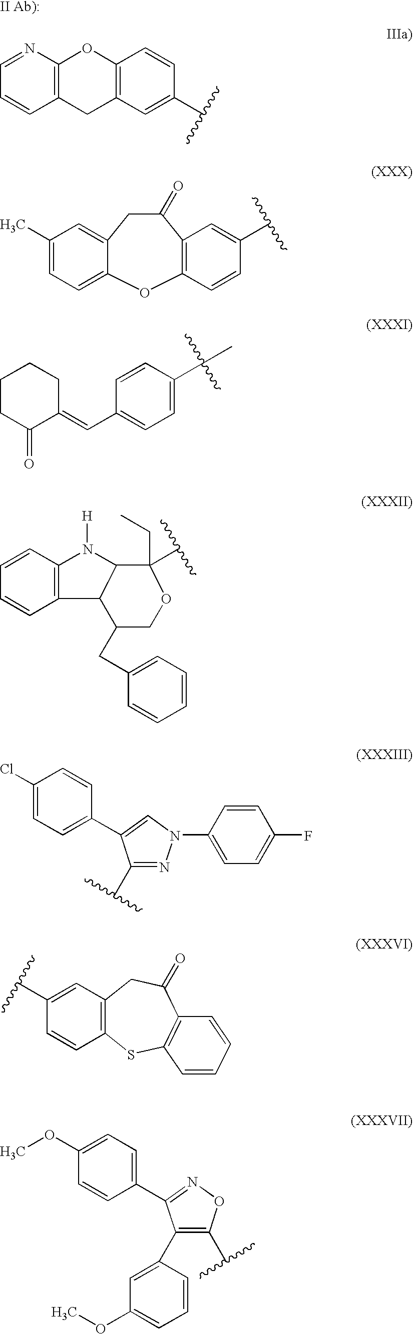
wherein:
when IIIa) contains —CH(CH₃)—COOH it is known as pranoprofen: α-methyl-5H-(1) benzopyran (2,3-b) pyridine-7-acetic acid;
when residue (XXX) contains —CH(CH₃)—COOH it is known as bermoprofen: dibenz(b,f)oxepin-2-acetic acid;
residue (XXXI) is known as CS-670: 2-(4-2(2-oxo-1-cyclohexylidenemethyl)phenyl)propionic acid, when the radical is —CH(CH₃)—COOH;
when residue (XXXII) contains group —CH₂COOH it is known as pemedolac;
when residue (XXXIII) is saturated with —CH₂COOH it is known as pyrazolac: 4-(4-chlorophenyl)-1-(4-fluorophenyl) 3-pyrazolyl acid derivatives;
when residue (XXXVI) is saturated with —CH(CH₃)—COO— it is known as zaltoprofen;
when residue (XXXVII) is CH₂—COOH it derives from the known mofezolac: 3,4-di p-methoxyphenyl)isoxazol-5-acetic acid;
Group IIIA), where t=1, 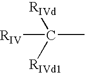
wherein:
at least one of R_{Ivd} and R_{Ivd1} is H and the other a linear or branched C₁-C₆ alkyl, or difluoroalkyl with the alkyl having from 1-6 C atoms, or R_{Ivd} and R_{Ivd} jointly form a methylene group;
R_{IV} has the following structure: 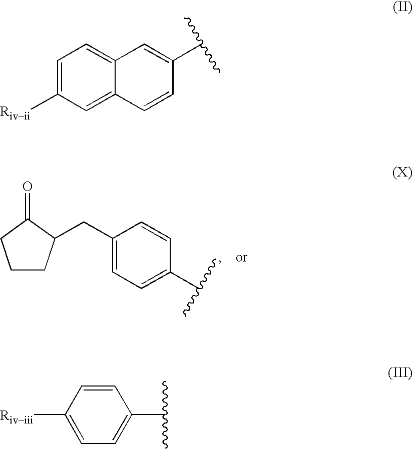
where:
in the residue of formula (II)):
R_{IV-II} is selected from the group consisting of an alkyl having from 1 to 6 C atoms, a cycloalkyl having from 3 to 7 C atoms, an alkoxymethyl having from 1 to 7 C atoms, a trifluoroalkyl having from 1 to 3 C atoms, vinyl, ethynyl, halogen, an alkoxy having from 1 to 6 C atoms, a difluroalkoxy with the alkyl having from 1 to 7 C atoms, an alkoxymethyloxy having from 1 to 7 C atoms, an alkylthiomethyloxy with the alkyl having from 1 to 7 C atoms, an alkylmethylthio with the alkyl having from 1 to 7 C atoms, cyano, difluoromethylthio, a substituted phenyl-, and phenylalkyl with the alkyl having from 1 to 8 C atoms;
R_{IV-II}, is a C₂-C₅ alkyl, a C₂ or C₃ alkyloxy, allyloxy, phenoxy, phenylthio, a cycloalkyl having from 5 to 7 C atoms, optionally substituted at position 1 by a C₁-C₂ alkyl;
Group IV A) 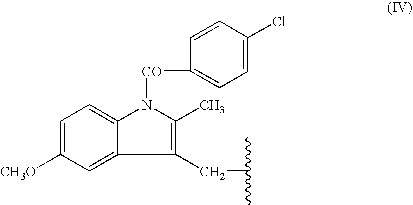
where A=RCOO, t=1,
Group V A) chosen from the following:
Subgroup V Aa) residues chosen from the following, where t=1 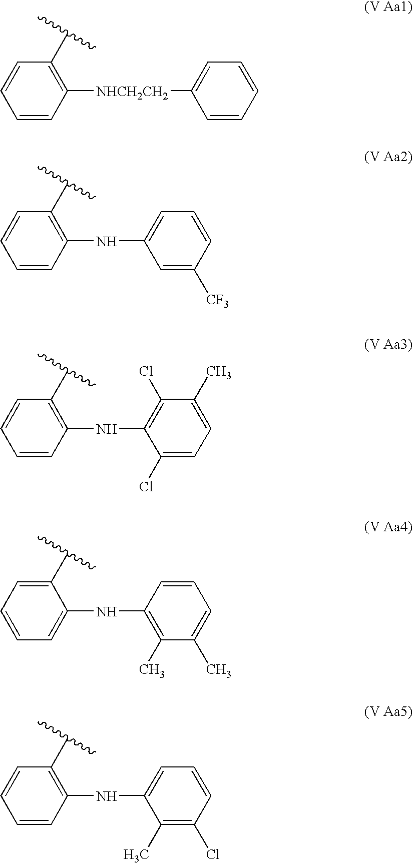
subgroup V Ab), residue, where t=1: 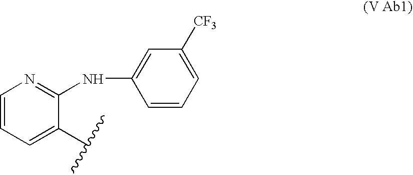
subgroup V Ac), residue, where t=0 and R is as follows: 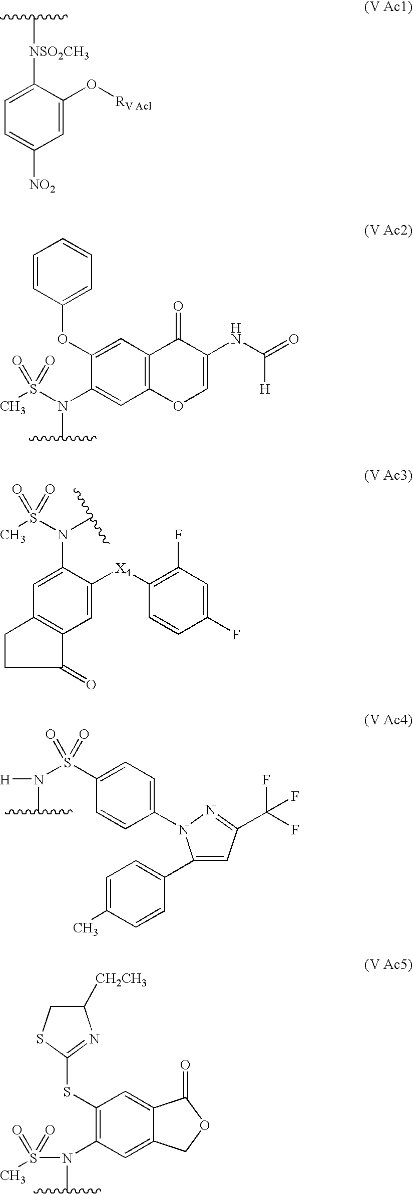
Subgroup V Ad) residues, where t=1 and R is as follows: 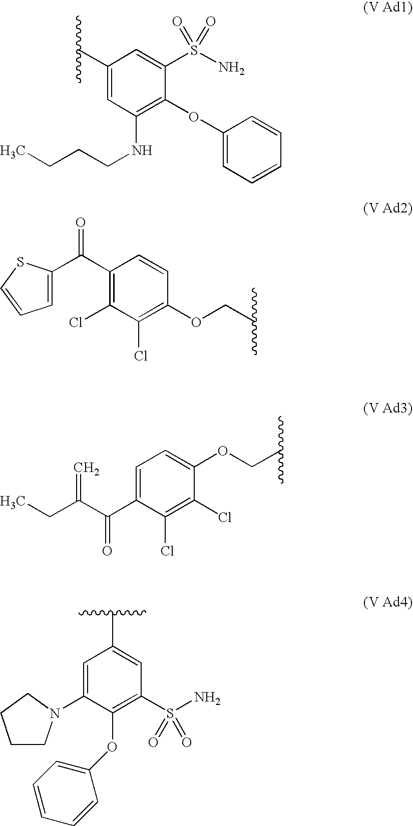
subgroup Ae) resides, where t=1 and R is as follows: 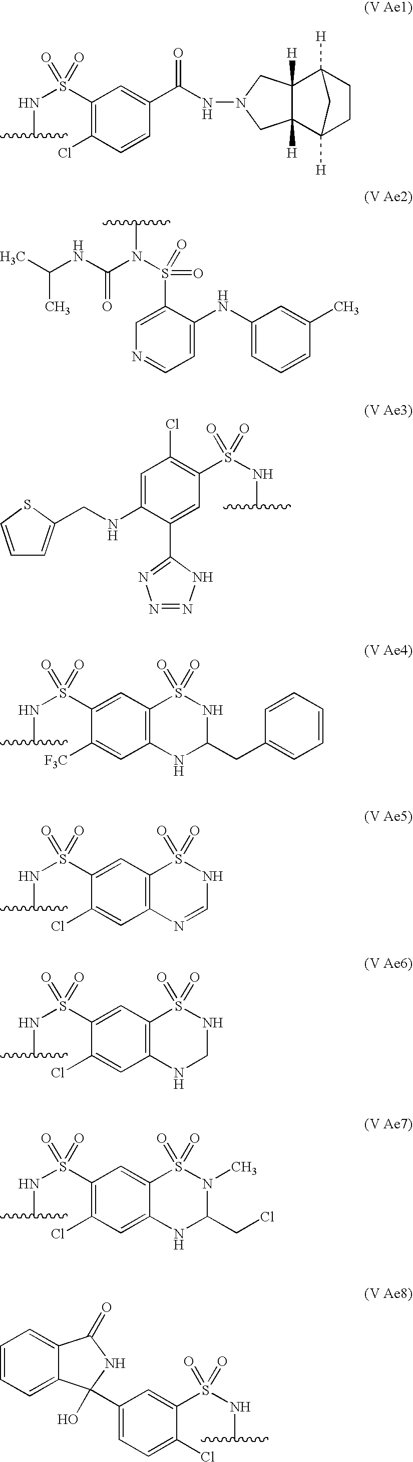 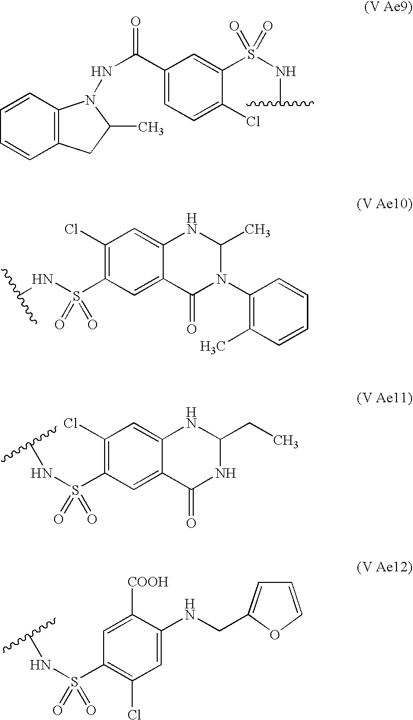
wherein:
in compounds (V Ac1) Rvac1 attached to the oxygen atom in position 2 of the benzene ring of the N-(4-nitro-phenyl)methansulphonamide can be phenyl or cyclohexane, when Rvac1 is phenyl the residue is that of nimesulfide;
in compounds (V Ac2) the residue of 3-formylamino-7-methylsulfonylamino-6-phenoxy-4H-1-bezopyran-4-one has been shown;
in compounds (V Ac3) the atom X₄ that links the radical 2,4-difluorothiophenyl to position 6 of the indanone ring of the residue 5-methanesulfonamido-1-indanone can be sulfur or oxygen;
Group VIA), where t=1, 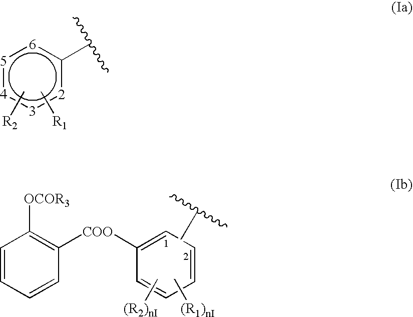
where: R₁ is group OCOR₃; where R₃ is methyl, ethyl or a linear or branched C₃-C₅ alkyl, or the residue of a single-ring heterocycle having 5 or 6 atoms which can be aromatic, partially or totally hydrogenated, containing one or more heteratoms independently chosen from O, N and S; R₂ is hydrogen, hydroxy, halogen, a linear or whenever possible branched alkyl having from 1 to 4 C atoms, a linear or whenever possible branched alcoxyl having from 1 to 4 C atoms; a linear or whenever possible branched perfluoroalkyl having from 1 to 4 C atoms, for example trifluoromethyl, nitro, amino, mono- or di(C₁₋₄)alkylamino;
R₁ and R₂ jointly are the dioxymethylene group, with the proviso that when X═NH, then X₁ is ethylene and R₂═H; R₁ cannot be OCOR₃ at position 2 when R₃ is methyl; nI being an integer from 0 to 1;
X₁ in formula A-X₁—NO₂ is a bivalent connecting bridge chosen from the following:
YO where Y is a linear or branched C₁-C₂₀ alkylene, or an optionally substituted cycloalkylene having from 5 to 7 carbon atoms; 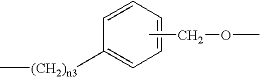
where n₃ is an integer from 0 to 3; 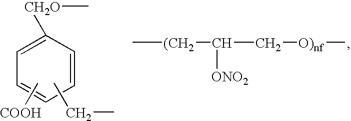
where nf′ is an integer from 1 to 6; 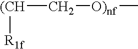
where R₁₁═H or CH₃ and nf is an integer from 1 to 6.

2. The method according to claim 1, in which R is selected from groups IIA) and VIA).

3. The method according to claim 1, in which R is as defined by group IIA), wherein R₃ₐ═H, R₂ₐ═CH₃, R₁ₐ is the formula (IX) and X═O.

4. The method according to claim 1, in which R is as defined by group VIA) (formula Ia), wherein R₁ is the group OCOR₃ with R₃═CH₃, R₂═H and X═O; R₁ is in the ortho position to CO.

5. A method for treatment of gastrointestinal tumors, according to claim 1, by administering compounds having the following formulas:

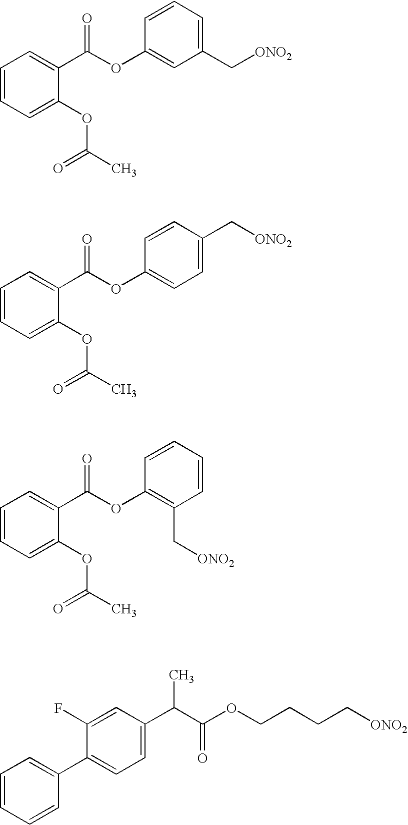

6. Use of compounds from groups IA) to VIA) for the treatment of gastrointestinal tumors.
